(19) United States
(12) Patent Application Publication
(10) Pub. No.: US 2005/0208156 A1
(43) Pub. Date: Sep. 22, 2005

(54) Plants extracts and the use thereof

(75) Inventors: Michael Ploch, Berlin (DE); Harald Murck, Berlin (DE); Thomas Haffner, Berlin (DE)

Correspondence Address: DILWORTH & BARRESE, LLP, 333 EARLE OVINGTON BLVD., UNIONDALE, NY, 11553, US

(73) Assignee: Lichtwer Pharma GmbH, Berlin (DE)

(21) Appl. No.: 10/509,261

(22) PCT Filed: Mar. 25, 2003
(86) PCT No.: PCT/EP03/03097

§ 371 Date: May 20, 2005

(30) Foreign Application Priority Data
Mar. 26, 2002 (DE) 102 13 571.1

(51) Int. Cl. A61K 35/78

(52) U.S. Cl. 424/728; 424/730; 424/753; 424/752

(57) ABSTRACT

The invention relates to the novel use of plants and plant extracts for treating schizophrenia. Said plants and plant extracts are selected from St John's wort, gingko, saffron and ginseng. The use of said substances occurs preferably in conjunction with conventional psychotherapeutic drugs, i.e. in the form of a preparation combining standard medicaments for treating schizophrenia for the simultaneous, separate or graduated use in case of schizophrenia. The invention also relates to methods for treating schizophrenia.

The present invention relates to a novel use of plants and parts of plants for the treatment of schizophrenia. The present invention relates in particular to the use of St John's wart, gingko, saffron and ginseng for the treatment of schizophrenia and the negative symptoms occurring therewith.

### PRIOR ART

The expression schizophrenia stems from Bleuler (1911) (G. Huber “Psychiatrie”, Schattauer Verlag, 5^{th} Edition, 1994), who shaped the modern disease concept and described the degeneration in the desires, feelings and actions of a patient suffering from this illness. In the acute phase, so-called positive symptoms prevail, such as 1. perceptual disturbances, for instance auditory hallucinations of voices that can be of a commentarial or imperative nature, 2. delusions, for instance delusions of persecution, 3. thought disorders such as, for instance, breaks in thought ranging to completely incoherent thought. These disorders have to persist for more than 4 weeks in order to be able to diagnose schizophrenia.

A complete restoration of health can also using today's medication only be achieved in approximately 1/3 of patients. The expression “Dementia praecox”, which was introduced for this group of patients before Bleuler by Kraepelin, the father of modern psychiatric classification, deems the core symptom to be the “premature” onset of the degradation of cognitive abilities. In this diagnosis, Kraepelin, contrary to Bleuler, considered the bad prognosis to be decisive for these patients. Thus, so-called negative or deficit symptoms were of more importance to him. Negative or deficit symptoms are cognitive impairments, apathy, blunted affect, lack of initiative, paucity of speech, and also reduced social capacities and neglect of personal hygiene. These symptoms in particular lead to considerable subsequent costs. Schizophrenia is a psychiatric disease which affects approximately 1% of the population over its lifetime. It first appears in 80% of those affected before the age of 40. The yearly costs owing to a loss in productivity and the resulting treatment costs in the USA at the beginning of the Nineties were approximately 30 to 50 billion Dollars. For individual persons, the illness means an approximately 20% lower life expectancy, in particular 10% of all persons affected by the illness commit suicide.

Schizophrenia is delimited over depression, an indication for which, for example, St John's wart is approved as an example of a plant extract, by means of its symptoms, however transitional forms and mixed states are also possible. The general criteria for diagnosing schizophrenia are defined according to the internationally valid classification scheme ICD 10 (International Classification of Mental Disorders, 10^{th} Edition, published by the World Health Organisation) in points F20.0 to F20.3: According hereto, schizophrenia can be diagnosed

if at least one symptom of symptom groups 1 to 4, namely: 1. thought echo, thought insertion, thought withdrawal and thought broadcasting; 2. delusions of control, delusions of influence, passivity, delusional perception; 3. commentarial or dialogic voices and 4. unrealistic and culturally inappropriate delusions, is clearly detectable or at least 2 symptoms of groups 5 to 8, namely 5. persistent hallucinations in any modality; 6. breaks or interference in the train of thought resulting in incoherence, irrelevant speech or neologisms; 7. catatonic symptoms; and 8. negative symptoms such as blunting of affect, paucity of speech and apathy are detectable almost all of the time over a period of one month or longer.

The acute forms of schizophrenia, i.e. paranoid schizophrenia (F 20.0), hebephrenic schizophrenia (F 20.1), catatonic schizophrenia (F 20.2) and undifferentiated schizophrenia (F 20.3), are identified using these criteria. The more precise differentiation between these forms is not important in this regard.

Patients thereby suffer from severe anxiety. They often feel that they are being remote controlled and that they no longer have any influence on their own desires and feelings. Noticeable for an outsider is the reaction of the affected person to their auditory hallucinations, in particular to voices which the patient sometimes answers but which can also lead to unpredictable actions by the patient. Of particular relevance thereby are acts that can lead to the death of the patient, for instance by jumping out of a window because they received the command to do so by a voice. Acute schizophrenia with the so-called positive symptoms occurring therewith is the classic mental disorder and is thus to be delimited over depression which is the classic affective disorder.

Negative or deficit symptoms have already been addressed above and are, for instance, cognitive impairments, apathy, blunted affect, lack of initiative, paucity of speech, and also reduced social capacities and neglect of personal hygiene. These symptoms occur in particular in the case of residual schizophrenia (ICD 10: F 20.5) or in simple schizophrenia (ICD 10: F 20.6), which are delimited over the forms of schizophrenia 20.0 to 20.3 cited above.

In order to be able to diagnose residual schizophrenia (ICD 10: F 20.5), a state of acute schizophrenia with the thereby prominent positive symptoms must have previously existed, however these symptoms are explicitly excluded for the diagnosis of simple schizophrenia. In the case of depression, which classically occurs in episodes but which can also become chronic, there is no previous history of acute schizophrenia having the criteria specified above either.

The following features are defined as further features of residual schizophrenia:

The occurrence of at least 4 of the following “negative symptoms” over the preceding twelve months:
1. psychomotor slowing or reduced activity,
2. clear blunting of affect,
3. passivity and lack of initiative,
4. diminution in the quantity or content of speech,
5. little non-verbal communication, notably by facial expression, eye contact, voice modulation and posture,
6. reduced social performance and neglect of personal hygiene.

These patients very often experience social decline and it is therefore not uncommon to find them amongst the homeless.

The same is true for patients with simple schizophrenia. The diagnostic criteria are as follows according to ICD 10:

Progressive development in all of the three following features over a period of at least one year:
1. clear and sustained changes in several earlier personality traits, manifested as a loss of drive and interest and as futile and aimless behaviour, self-absorption and social withdrawal,
2. gradual occurrence and intensification of “negative” symptoms such as apathy, paucity of speech, reduced activity, clear blunting of affect, passivity, lack of initiative and reduced non-verbal communication (facial expressions, eye contact, voice modulation or posture),
3. clear reduction in academic and professional performance.

The symptoms of the two disorders of simple schizophrenia and residual schizophrenia are similar to a large extent. These symptoms can be delimited over depression in particular owing to the lack of a prominent depressed mood as opposed to the blunting of affect in this case, as well as owing to the different temporal progression of the disorders.

The following concepts have been developed and used to date for the therapy of schizophrenia.

Following the introduction of synthetic neuroleptics in the 1950's, positive symptoms were predominantly of interest since these could be treated well with these new substances. The first synthesised neuroleptic chlorpromazine is a phenothiazine derivate and the precursor of a whole group of similar substances. Another substance class including butyrophenones such as, for example, haloperidol, was also developed for this indication. Common to all these substances is a blocking property at certain dopamine receptors. They are summarised as “classic neuroleptics”.

The first synthetic antidepressant imipramine was developed several years later. This is a so-called tricyclic antidepressant whose mechanism of action lies in inhibiting the reuptake of certain neurotransmitters, namely noradrenaline and serotonin. Most of the synthetic antidepressants currently used are based on this property of the substances. Differing from neuroleptics, antidepressants are not effective for the treatment of acute schizophrenia.

The deficit symptoms only became a focus of interest again with the introduction of the first so-called atypical neuroleptic clozapine at the end of the Seventies since clozapine also has a clear influence thereon. This is in particular the case because in addition to clozapine, which was the only atypical neuroleptic available for a long time, a whole range thereof in the meantime exists, as shown in a review by Möller (Möller H-J, (2000): Nervenarzt 71:345-353). The following is stated in this review with regard to the question of negative symptoms: “a clinically very significant question because chronic negative symptoms essentially determine the course of schizophrenic disorders”. Main advertising messages of the new substances are also directed at efficiency in the case of negative symptoms. There are, however, several differences between these new substances both with regard to their profile of action and to their side effect profile. Taking clozapine is linked with the risk of agranulocytosis, i.e. the disappearance of certain white blood cells, and is therefore provided with clear regulations on the part of the distributing company. Both clozapine and olanzapine lead to significant weight gain, to a lesser extent this is also true for risperidone. Möller writes in this regard: “This, in some cases, considerable weight gain and, inter alia, the medical consequences thereof will probably have a central position in the side—effect problems of neuroleptics in the future”.

The object of the present invention is thus to provide a medicament which does not have the disadvantages cited above and which treats schizophrenia, in particular the negative symptoms of schizophrenia.

### BRIEF DESCRIPTION OF THE INVENTION

The present invention relates to the use of plants and plant extracts for the treatment of schizophrenia. The present invention relates in particular to the use of St John's wart, gingko, saffron and ginseng for the treatment of schizophrenia. Plant extracts of the aforementioned fresh and dried plants are particularly suitable therefor.

These plant extracts are preferably alcoholic or alcohol aqueous extracts.

The substances are used according to the invention in conventional pharmaceutical forms of administration such as liquid, semi-solid and solid forms.

The invention furthermore relates to the combined use of the aforementioned substances with conventional therapeutic agents in the treatment of schizophrenia. This means that the substances can be used as a combination preparation with conventional medicaments for treating schizophrenia for the simultaneous, separate or graduated use in the case of schizophrenia.

The present invention is finally directed at methods for treating individuals suffering from schizophrenia.

### DETAILED DESCRIPTION OF THE INVENTION

The new indication for St John's wart, gingko, saffron and ginseng is the treatment of schizophrenia and in particular the treatment of negative symptoms, particularly in patients with chronic schizophrenia. The medication can thereby be provided as an add-on therapy to a therapy with a conventional psychotherapeutic drug for treating schizophrenia, such as a neuroleptic, i.e. inter alia as a combination preparation with conventional neuroleptics or other psychotherapeutic drugs used for treating schizophrenia.

According to one embodiment, the additional administration of, for example, St John's wart extract to a basic medication of a classic neuroleptic is thereby advantageous, in particular for patients who have been taking this for a long period of time and who are, on the whole, psychopathologically stable.

Substances such as haloperidol, benperidol, chlorprotixene, flupentixol, fluphenazine, perazine, perphenazine and thioridazine in particular can be mentioned as classic neuroleptics. Add-on therapy is, however, also possible for a basic therapy with atypical neuroleptics such as, clozapine, olanzapine, seroquel, sertindole, and also all other psychotherapeutic drugs suitable for treating schizophrenia.

This means that pursuant to the invention, in accordance with one embodiment, a combination preparation is provided that contains St John's wart, gingko, saffron and/or ginseng in addition to, as further components, a conventional psychotherapeutic drug against schizophrenia for simultaneous, separate or graduated use in the treatment of schizophrenia.

This combination preparation or a simple combination of the claimed plant preparations with psychotherapeutic drugs for treating schizophrenia, such as neuroleptics, in particular from the group of classic neuroleptics such as haloperidol, benperidol, chlorprotixene, flupentixol, fluphenazine, perazine, perphenazine and thioridazine, can be used for the add-on therapy. The add-on therapy is, however, also possible for a basic therapy with atypical neuroleptics such as, clozapine, olanzapine, seroquel, sertindole, and also all other psychotherapeutic drugs suitable for treating schizophrenia.

St John's wart extract, for example, should not be administered if florid psychotic symptoms such as perceptual disorders or delusions exist.

St John's wart (Hypericum perforatum) can be present in fresh or dried form. All parts of the plant, depending on the stage of growth, can be used for direct application and for obtaining extracts, preferably the parts above ground such as, for example the flowers, leaves, stalk and seed pods. Typical components are hypericins, hyperforins, flavonoids and bioflavones.

Ginkgo (Ginkgo biloba) can be present in fresh, preferably dried form. The leaves are preferably used for direct application and for obtaining extracts. Typical components are flavone glycosides and terpene lactones, for example bilobalide, ginkgolides.

Saffron (Crocus sativus) can be present in fresh or dried form. All parts of the plant, depending on the stage of growth, can be used for direct application and for obtaining extracts, preferably the parts above ground, in particular the stigmas. Typical components are α-, β-pinene, 1,8 cineol, the carotenoid crocin, picrocrocin as well as the degradation product thereof safranal.

Ginseng (Panax ginseng) can be present in fresh or dried form. All parts of the plant, depending on the stage of growth, can be used for direct application and for obtaining extracts, preferably the roots. Typical components are triterpene saponines (ginsenosides), sesquiterpenes and polyacetylenes.

The extraction agent used in the one- or multi-step method according to the invention is preferably a non-aqueous extraction agent such as an organic solvent or an aqueous alcoholic extraction agent. Cited here as examples are alcohols, ketones, esters, ethers, aromatics, halogenated compounds, alkanes, alkenes and the like, either in pure form or as mixtures with water. Aliphatic alcohols, ketones and carboxylic acid esters are particularly suitable. These solvents can be used alone or as mixtures of the compounds cited above. Supercritical and subcritical gases such as, for example, carbon dioxide (CO₂) and ethane are also usable extraction agents, also mixed with the compounds cited above. Pure supercritical carbon dioxide or mixed with further solvents such as, for example, methanol are cited as an example.

The extract used can be an alcoholic, alcoholic-aqueous extract with primary, secondary and tertiary alcohols of the series C1 to C5, preferably methanol and ethanol, in the composition of alcohol/water of between 100/0 and 30/70, preferably 80/20 to 50/50.

In a particularly preferred embodiment, 80% methanol (20% water) is used as the extraction agent for St John's wart, 60% acetone (40% water) is used for gingko, also in conjunction with reprecipitation steps and subsequent extraction steps for example using ketones, butanol and toluene, >90% ethanol (<10% water) is used for saffron, and 30% ethanol (70% water) is used for ginseng.

The extracts are obtained from the fresh or dried initial material using extraction methods without agitation such as maceration and extraction methods with agitation such as percolation, ultra turrax extraction and ultrasound extraction (liquid-liquid method). They are present directly as liquid extracts or as concentrated spissum extracts or in the form of their dried extracts. Dry extracts are preferred for St John's wart, gingko and ginseng, and liquid or spissum extracts are preferred for saffron. St John's wart extracts preferably contain hypericins, hyperforins and flavonoids, particularly preferred in the following concentrations:
0.01-2% hypericins, 0.01-30% hyperforins, 2-35% flavonoids, preferably 0.10 to 0.40% hypericins and 1-6% hyperforins.

Extracts of Gingko biloba leaves contain flavone glycosides and terpene lactones, particularly preferred in the following concentrations: 20 to 30% by weight flavone glycosides, together with 2-8% ginkgolides, particularly preferred 23 to 27% flavone glycosides and 5 to 7% by weight ginkgolides.

Saffron extracts contain, inter alia, α-, β-pinene, 1,8 cineol, crocin, picrocrocin and optionally the degradation product thereof safranal, particularly preferred in the following concentrations: 5-10% pinene and cineol, 4-10% picrocrocin and/or 2-6% safranal. Ginseng root extracts contain, inter alia, triterpene saponines (ginsenosides/ginsenoids), sesquiterpenes and polyacetylenes, particularly preferred in the following concentrations: 3-9% ginsenoids.

The described substances of the aforementioned plants can be processed into and applied as conventional solid, semi-solid and liquid pharmaceutical and other forms of administration, for example in powders, solutions, suspensions, tablets, film-coated tablets, dragees, capsules, effervescent tablets, effervescent granulate, chew tablets, lozenges, suppositories, creams, salves and gels. Conventional auxiliary agents can thereby be used for the respective form of administration, for example celluloses, silicon dioxides, lactose, natural and synthetic polymers, salts, colorants, flavouring agents, fats, oils, surfactants, water and alcohols.

The daily dose of St John's wart extract should thereby be in the range of 300 mg of extract to 2700 mg of extract, preferably 1500 mg of extract divided into two individual doses.

A dose ranging from 50 mg of extract to 1000 mg of extract is preferred for gingko extract, saffron extract and ginseng extract. The condition of the individual, for example age, weight, etc, of course also plays a role when determining the dose.

A method for treating schizophrenia is furthermore provided according to the invention. This method includes the administration of Hypericum perforatum (St John's wart) and/or Ginkgo biloba (gingko) and/or Crocus sativus (saffron) and/or Panax ginseng (ginseng) to individuals suffering from schizophrenia.

In particular the plant, plant parts, dried plant or plant parts, extracts, extract fractions, pure substances and their derivatives and the salts of the plant are thereby used for administration to individuals for treating schizophrenia.

The extracts of the individual plants described above are particularly advantageous. These can be administered either alone or in combination in the treatment method.

The treatment method according to the invention also includes the simultaneous, separate or graduated administration of the aforementioned active substances with a conventional psychotherapeutic drug against schizophrenia for the treatment of schizophrenia.

The treatment method particularly advantageously relates to an add-on therapy of the aforementioned active substances in combination with psychotherapeutic drugs for treating schizophrenia, in particular neuroleptics, in particular of the group of classic neuroleptics such as haloperidol, benperidol, chlorprotixene, flupentixol, fluphenazine, perazine, perphenazine, thioridazine, atypical neuroleptics, in particular clozapine, olanzapine, seroquel, sertindole, as well as other psychotherapeutic drugs suitable for treating schizophrenia.

### EXAMPLE

A well-established model of cognitive disorders in patients with chronic schizophrenia is the induction of thought disorders owing to the administration of the anaesthetic ketamine {Duncan G. et al (2000) J Pharmacol Exp Ther 293:8-14, Krystal J H, et al (2000): Biol Psychiatry 47:137-143}, which are reduced by atypical neuroleptics such as olanzapine and clozapine {Duncan, ibid.}. ST JOHN'S WART EXTRACT showed an antagonism on the effect of ketamine on the induction of sleep in an examination using animals (Butterweck V. et al. (1997): Pharmacopsychiatry 30 Suppl 2: 117-124).

In the following, the ketamine-antagonistic effect of hypericum extract was examined as a parameter that shows a possible effectivity of St John's wart on the negative symptoms of patients with chronic schizophrenia. It was furthermore examined whether the subchronic administration of 2×750 mg of St John's wart extract (Jarsin® 750 mg, Lichtwer Pharma AG, Berlin, Germany) has an effect on changes induced by ketamine after 7 days of taking one tablet orally in the morning and the evening. This study was carried out with a double-blind, randomised design on 16 healthy test persons. A so-called “cross-over design” was thereby used: half of the test persons received a placebo for one week, then there was one week without medication and then St John's wart extract was administered for one week. The other half received St John's wart extract in the first week and the placebo in the last week. The test persons were assigned randomly to groups.

The change of electrophysiologically measured values owing to the administration of ketamine was selected as a parameter and the change in cognitive abilities was furthermore examined in a special test.

In detail, the change in the difference of the peak N1-P2 of the auditory evoked potential (AEP) was consulted as the primary target parameter. A stimulus of 100 ms duration having a frequency of 1 kHz was thereby played in a random manner to an ear via a headphone. The loudness of the tone was constant at 90 dBA and the intervals between the tones varied between 4 and 8 seconds. Since AEP's are not subject to habituation, they are well suited to an examination in the cross-over design.

The change of further parameters to be determined electrophysiologically was selected as a secondary target parameter, namely the parameters of latency, of angular velocity, of time until fixation, of the reaction time until a selection occurs and of the number of correct reactions in the occulodynamic test (ODT). The ODT delivers parameters of the central nervous system capacity, which are not influenced by changed motivation or learning. They are therefore also well suited to a cross-over design. In the ODT, light signals are presented in a random order at defined horizontal positions of the visual axis. A total of 900 signals are thereby used within 15 minutes. The eye movements can be measured by means of an electroocculogram (EOG) and the following parameters can be obtained:
1. average latency between the appearance of the light signal and the start of eye movement,
2. average angular velocity of eye movement,
3. time until fixation.

Furthermore, the signal identification exercise, which represents a complex selection reaction and is also a measure for cognitive ability, provides other parameters, in particular the number of correct reactions and the average reaction time.

Further secondary target parameters were determined using a special cognitive test, namely the SKT test (Lehfeld and Erzigheit, Int. Psychogeriatr. 1997; 9 Suppl 1: 115-21).

The change in the electrophysiological examinations as well as in the cognitive test owing to ketamine was carried out at the end of each of the one-week treatment periods. First of all examination occurred under baseline conditions. Then 4 mg of ketamine was infused over a period of one hour. 30 minutes after the start of infusion, the AEP's were measured again and the ODT and the SKT test were carried out.

The AEPs showed a clear reduction in the N1-P2 peak amplitude in the placebo condition. This reduction was completely abolished by St John's wart extract, and thus there was a highly significant effect for the effectivity of St John's wart extract (p=0.004) (table 1).

TABLE 1
2 × 2 Cross-Over Analysis
Parameter: change (difference before/after ketamine) to AEP parameters
(primary target variable)

| Parameter (μV) | Medication | Value | Standard Error |
|---|---|---|---|
| Change of the N1/P2 | Placebo | −8.3946 | 3.8947 |
| peak-to-peak amplitude | | | |
| | Verum | 10.3174 | 3.8947 |
| Change of the N1 | Placebo | −4.9315 | 1.8671 |
| amplitude | | | |
| | Verum | 3.6559 | 1.8671 |
| Change of the P2 | Placebo | −3.4631 | 2.2432 |
| amplitude | | | |
| | Verum | 6.6615 | 2.2432 |

Difference between Placebo and Verum

| Parameter (μV) | Value | Standard Error | p |
|---|---|---|---|
| Change of the N1/P2 | −18.7121 | 5.5080 | 0.0040 |
| peak-to-peak amplitude | | | |
| Change of the N1 | −8.5874 | 2.6405 | 0.0054 |
| amplitude | | | |
| Change of the P2 | −10.1246 | 3.1724 | 0.0061 |
| amplitude | | | |

The same is also true for the individual components N1 and P2. Under baseline conditions, i.e. before ketamine is administered, there was no significant difference between the placebo and the verum. This shows that the described effect must be regarded as ketamine antagonism.

In the ODT, there was no difference in the number of correct answers between the placebo group and the verum group. All other parameters, namely the selection reaction time, the angular velocity and the latency showed a trend towards impairment owing to ketamine and a distinct reduction in impairment owing to St. John's wart extract.

In the SKT test, the impairments owing to ketamine were small and inhomogeneous, and thus no difference could be observed between the placebo and the verum.

The represented findings show that taking 2×750 mg of hypericum extract has a ketamine antagonistic effect. It is therefore clear that this medication can have a therapeutic effect on the negative symptoms of schizophrenia.

The same effects could also be observed when using gingko, saffron and ginseng.

1-13. (canceled)

14. Use of Hypericum perforatum (St. John's wart) and/or Ginko biloba (gingko) and/or Crocus sativus (saffron) and/or Panax ginseng (ginseng) for the production of a medicament for the treatment of schizophrenia.

15. Use according to claim 14, wherein the plant, plant parts, dried plant or plant parts, extracts, extract fractions, pure substances and their derivatives and the salts of the plant are used.

16. Use according to claim 14, wherein the extract used is an alcoholic, alcoholic-aqueous extract with primary, secondary and tertiary alcohols of the series C1 to C5, preferably methanol and ethanol, in the composition of alcohol/water of between 100/0 to 30/70, preferably 80/20 to 50/50.

17. Use according to claim 16, wherein there are produced in a one- and multi-step production process.

18. Use according to claim 14, wherein the St. John's wart extract used has the following amounts of components: 0.01-2% herpericins, 0.01-30% hyperforins, 2-35% flavonoids, preferably 0.10 to 0.40% hypericins and 1-6% hyperforins.

19. Use according to claim 14, wherein the gingko extract has the following amounts and components: 20-30% by weight flavone glycosides, together with 2-8% of ginkgolides, particularly preferred 23 to 27% flavone glycosides and 5 to 7% by weight ginkgolides.

20. Use according to claim 14, wherein the saffron extract comprises the components α-, β-pinene, 1,8 cineol, crocin, picrocrocin as well as optionally the degredation product thereof safranal, particularly preferred in the following concentrations: 5-10% pinene and cineol, 4-10% picrocrocin and/or 2-6% safranal.

21. Use according to claim 14, wherein the ginseng root extract comprises the components, inter alia, triterpene saponines (ginsenosides/ginsenoids), sesquiterpenes and polyacetylenes, particularly preferred in the following concentrations: 3-9% ginsenoids.

22. Use according to claim 14, wherein use occurs in the form of liquid, semi-solid and solid forms of administration, in particular solutions, suspensions, tablets, film-coated tablets, dragees, capsules, effervescent tablets, effercescent granulate, chew tablets and suppositories.

23. Use according to claim 14, wherein the case of St. John's wart extract, the daily dose of the extracts is 300 to 2700 mg in up to 3 separate doses per day, preferably 750-1500 mg in 1-2 separate doses per day.

24. Use according to claim 14, wherein in the case of gingko biloba, Crocis sativus and Panax ginseng, the daily dose of the extracts is 50 mg-1000 mg of extract.

25. Combination preparation containing St. John's wart, gingko, saffron and/or ginseng in addition to, as further components, a psychotherapeutic drug against schizophrenia for the simultaneous, separate or graduated use in the treatment of schizophrenia.

26. Use according to claim 14, in add-on therapy in combination with psychotherapeutic drugs for treating schizophrenia, in particular neuroleptics, in particular of the group of classic neuroleptics such as haloperidol, benperidol, chloroprotixene, flupentixol, fluphenazine, perazine, perphenazine, thioridazine, atypical neuroletics, in particular clozapine, olanzapine, seroquel, sertindole, as well as other psychotherapeutic drugs suitable for treating schizophrenia.

27. Use according to claim 15, wherein the extract used is an alcoholic, alcoholic-aqueous extract with primary, secondary and tertiary alcohols of the series C1 to C5, preferably methanol and ethanol, in the composition of alcohol/water of between 100/0 to 30/70, preferably 80/20 to 50/50.

28. Use according to claim 27, wherein there are produced in a one- and multi-step production process.

29. Use according to claim 15, wherein the St. John's wart extract used has the following amounts of components: 0.01-2% herpericins, 0.01-30% hyperforins, 2-35% flavonoids, preferably 0.10 to 0.40% hypericins and 1-6% hyperforins.

30. Use according to claim 16, wherein the St. John's wart extract used has the following amounts of components: 0.01-2% herpericins, 0.01-30% hyperforins, 2-35% flavonoids, preferably 0.10 to 0.40% hypericins and 1-6% hyperforins.

31. Use according to claim 17, wherein the St. John's wart extract used has the following amounts of components: 0.01-2% herpericins, 0.01-30% hyperforins, 2-35% flavonoids, preferably 0.10 to 0.40% hypericins and 1-6% hyperforins.

32. Use according to claim 27, wherein the St. John's wart extract used has the following amounts of components: 0.01-2% herpericins, 0.01-30% hyperforins, 2-35% flavonoids, preferably 0.10 to 0.40% hypericins and 1-6% hyperforins.

33. Use according to claim 28, wherein the St. John's wart extract used has the following amounts of components: 0.01-2% herpericins, 0.01-30% hyperforins, 2-35% flavonoids, preferably 0.10 to 0.40% hypericins and 1-6% hyperforins.
